(19) United States
(12) Patent Application Publication
(10) Pub. No.: US 2021/0202085 A1
(43) Pub. Date: Jul. 1, 2021

(54) APPARATUS FOR AUTOMATICALLY TRIAGING PATIENT AND AUTOMATIC TRIAGE METHOD

(71) Applicant: BOE TECHNOLOGY GROUP CO., LTD., Beijing (CN)

(72) Inventors: Jiantao Liu, Beijing (CN); Honglei Zhang, Beijing (CN); Xuewen Lv, Beijing (CN); Fang Liu, Beijing (CN)

(73) Assignee: BOE TECHNOLOGY GROUP CO., LTD., Beijing (CN)

(21) Appl. No.: 16/063,940

(22) PCT Filed: Dec. 14, 2017
(86) PCT No.: PCT/CN2017/116191

§ 371 Date: Jun. 19, 2018

(30) Foreign Application Priority Data
Jun. 28, 2017 (CN) 201710507456.0

(51) Int. Cl. G16H 40/67; G16H 10/20; G06K 9/00; G06K 9/62; G06F 40/30; G06F 40/40

(52) U.S. Cl. CPC G16H 40/67; G16H 10/20; G06K 9/00362; G06F 40/40; G06K 9/6276; G06K 9/6259; G06F 40/30; G06K 9/6223

(57) ABSTRACT

The present application discloses an apparatus for automatically triaging a patient. The apparatus includes a receiver configured to receive patient information of the patient from a terminal; a feature extractor configured to extract feature information from the patient information; a selector configured to provide a recommendation on a hospital and a department of the hospital for treating the patient based on the feature information extracted by the feature extractor, and a transmitter configured to transmit the recommendation to the terminal.

### CROSS-REFERENCE TO RELATED APPLICATION

This application claims priority to Chinese Patent Application No. 201710507456.0, filed Jun. 28, 2017, the contents of which are incorporated by reference in the entirety.

### TECHNICAL FIELD

The present invention relates to information automation technology, more particularly, to an apparatus for automatically triaging a patient and an automatic triage method.

### BACKGROUND

Triage. e.g., assigning patients to a specific department, is typically the first step a patient experiences in a hospital. A patient can only be properly treated if she or he is triaged to a department specializing in treating the disease she or he has. Incorrectly triaging a patient results in re-treatment of the patient, wastes of medical resources and the patient's time, and, in some cases, delay of proper treatment in the patient.

### SUMMARY

In one aspect, the present invention provides an apparatus for automatically triaging a patient, comprising a receiver configured to receive patient information of the patient from a terminal; a feature extractor configured to extract feature information from the patient information; a selector configured to provide a recommendation on a hospital and a department of the hospital for treating the patient based on the feature information extracted by the feature extractor; and a transmitter configured to transmit the recommendation to the terminal.

Optionally, the apparatus further comprises a data base storing a plurality of reference feature information; wherein the selector is configured to compare the feature information extracted from the patient information with the plurality of reference feature information, and provide the recommendation based on a result of comparing by the selector.

Optionally, the plurality of reference feature information comprises a plurality of reference feature information of patients treated by a plurality of hospitals and a plurality of departments of the plurality of hospitals; the selector is configured to select one of the plurality of reference feature information of patients treated in one of the plurality of hospitals and one of the plurality of departments as a closest match to the feature information extracted from the patient information, and recommend a selected hospital and a selected department having the closest match for treating the patient.

Optionally, the patient information comprises an image of a body part of the patient; the feature extractor is configured to extract a feature partial image information from the image of the body part of the patient as the feature information; the plurality of reference feature information comprise a plurality of reference feature partial image information; and the selector is configured to select one of the plurality of reference feature partial image information as the closest match to the feature partial image information, and recommend the selected hospital and the selected department having the closest match for treating the patient.

Optionally, the plurality of reference feature partial image information comprise feature partial image information extracted from images of body parts of patients treated in the plurality of hospitals and the plurality of departments.

Optionally, extracting the feature partial image information is performed by an image recognition technique; and the selector is configured to select the closest match using a classification algorithm.

Optionally, the classification algorithm comprises one or a combination of a k-means algorithm, and a learning vector quantization-based neural network classification algorithm.

Optionally, the patient information comprises an image of a diagnostic textual data; the feature extractor is configured to recognize a textual data from the image of the diagnostic textual data using a textual recognition technique, and extract a semantic feature information from the textual data as the feature information using a semantic analysis technique; the plurality of reference feature information comprise a plurality of reference semantic feature information; and the selector is configured to select one of the plurality of reference semantic feature information as the closest match to the semantic feature information, and recommend the selected hospital and the selected department having the closest match for treating the patient.

Optionally, the plurality of reference semantic feature information comprise semantic feature information extracted from diagnostic textual data of patients treated in the plurality of hospitals and the plurality of departments.

Optionally, extracting the semantic feature information is performed by a natural language processing technique.

Optionally, the apparatus further comprises a question generator configured to generate a health information query and send the health information query to the terminal; wherein the receiver is configured to receive an answer to the health information query from the terminal as the patient information of the patient.

Optionally, the selector is configured to provide the recommendation on a plurality of hospitals and a plurality of departments thereof for treating the patient, and rank the plurality of hospitals and the plurality of departments; and the transmitter is configured to transmit to the terminal information on one or more highest-ranking hospitals and one or more highest-ranking departments as the recommendation.

In another aspect, the present invention provides an automatic triage method, comprising receiving patient information of a patient from a terminal; extracting feature information from the patient information using a feature extractor; providing a recommendation on a hospital and a department of the hospital for treating the patient based on the feature information extracted by the feature extractor; and transmitting the recommendation to the terminal.

Optionally, the automatic triage method further comprises storing a plurality of reference feature information; comparing the feature information extracted from the patient information with the plurality of reference feature information; and providing the recommendation based on a result of comparing.

Optionally, the plurality of reference feature information comprises a plurality of reference feature information of patients treated by a plurality of hospitals and a plurality of departments of the plurality of hospitals; the method further comprises selecting one of the plurality of reference feature information of patients treated in one of the plurality of hospitals and one of the plurality of departments as a closest match to the feature information extracted from the patient information; and recommending a selected hospital and a selected department having the closest match for treating the patient.

Optionally, the patient information comprises an image of a body part of the patient; and the plurality of reference feature information comprise a plurality of reference feature partial image information; the method further comprises extracting a feature partial image information from the image of the body part of the patient as the feature information; selecting one of the plurality of reference feature partial image information as the closest match to the feature partial image information; and recommending the selected hospital and the selected department having the closest match for treating the patient.

Optionally, the plurality of reference feature partial image information comprise feature partial image information extracted from images of body parts of patients treated in the plurality of hospitals and the plurality of departments.

Optionally, extracting the feature partial image information is performed by an image recognition technique; and selecting the closest match is performed using a classification algorithm.

Optionally, the classification algorithm comprises one or a combination of a k-means algorithm, and a learning vector quantization-based neural network classification algorithm.

Optionally, the patient information comprises an image of a diagnostic textual data; and the plurality of reference feature information comprise a plurality of reference semantic feature information; the method further comprises recognizing a textual data from the image of the diagnostic textual data using a textual recognition technique; extracting a semantic feature information from the textual data as the feature information using a semantic analysis technique; selecting one of the plurality of reference semantic feature information as the closest match to the semantic feature information; and recommending the selected hospital and the selected department having the closest match for treating the patient.

Optionally, the plurality of reference semantic feature information comprise semantic feature information extracted from diagnostic textual data of patients treated in the plurality of hospitals and the plurality of departments.

Optionally, extracting the semantic feature information is performed by a natural language processing technique.

Optionally, the automatic triage method further comprises generating a health information query; sending the health information query to the terminal; and receiving an answer to the health information query from the terminal as the patient information of the patient.

Optionally, providing the recommendation comprises providing the recommendation on a plurality of hospitals and a plurality of departments thereof for treating the patient, and ranking the plurality of hospitals and the plurality of departments; and transmitting the recommendation to the terminal comprises transmitting to the terminal information on one or more highest-ranking hospitals and one or more highest-ranking departments as the recommendation.

### BRIEF DESCRIPTION OF THE FIGURES

The following drawings are merely examples for illustrative purposes according to various disclosed embodiments and are not intended to limit the scope of the present invention.

FIG. 1 is a schematic diagram illustrating the structure of an apparatus for automatically triaging a patient in some embodiments according to the present disclosure.

FIG. 2 depicts a tongue image and disease correlation with feature information of different parts of the tongue image.

FIG. 3 is a flow chart illustrating an automatic triage method in some embodiments according to the present disclosure.

### DETAILED DESCRIPTION

The disclosure will now be described more specifically with reference to the following embodiments. It is to be noted that the following descriptions of some embodiments are presented herein for purpose of illustration and description only. It is not intended to be exhaustive or to be limited to the precise form disclosed.

Typically, the patient herself or himself decides where to get diagnosis and treatment for her or his conditions or diseases. For example, the patient can make the decision based on her or his symptoms or based on results of consulting with an information desk in a hospital. With the development of the medicine, the medical practice becomes more and more specialized. For example, internal medicine alone can have more than ten sub-specialties such as allergy and immunology, cardiovascular diseases, endocrinology, diabetes, and metabolism, and so on. Typically, the patient has only very limited medical knowledge. It is very difficult for the patient to make accurate judgment on her or his own as to from which department and sub-specialty she or he should seek treatment, even with the help from the staff member at the information desk of a hospital.

Moreover, most hospitals have their own specialties. If the patient goes to a hospital which lacks the specialty for treating the patient's condition or disease, the patient will have to seek treatment from another hospital, resulting waste of the patient's time.

Accordingly, the present disclosure provides, inter alia, an apparatus for automatically triaging a patient and an automatic triage method that substantially obviate one or more of the problems due to limitations and disadvantages of the related art. In one aspect, the present disclosure provides an apparatus for automatically triaging a patient. In some embodiments, the apparatus includes a receiver configured to receive patient information of the patient from a terminal; a feature extractor configured to extract feature information from the patient information; a selector configured to provide a recommendation on a hospital and a department of the hospital for treating the patient based on the feature information extracted by the feature extractor; and a transmitter configured to transmit the recommendation to the terminal.

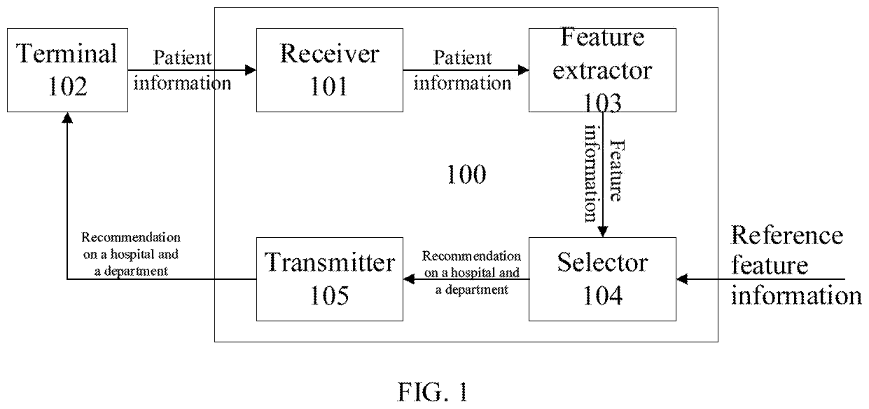

FIG. 1 is a schematic diagram illustrating the structure of an apparatus for automatically triaging a patient in some embodiments according to the present disclosure. Referring to FIG. 1, the apparatus 100 for automatically triaging a patient in some embodiments includes a receiver 101 configured to receive patient information of the patient from a terminal 102; a feature extractor 103 configured to extract feature information from the patient information a selector 104 configured to provide a recommendation on a hospital and a department of the hospital for treating the patient based on the feature information extracted by the feature extractor 103; and a transmitter 105 configured to transmit the recommendation to the terminal 102.

In some embodiments, the receiver 101 is configured to receive patient information of the patient from a terminal 102 by a wire or wirelessly. e.g., via internet or a wireless communication means (e.g., Bluetooth). The terminal 102 may be, for example, a computer or a mobile phone of the patient. Optionally, the patient information includes patient health information such as information indicating one or more health conditions of the patient. The feature extractor 103 extracts from the patient information feature information useful for triaging the patient. The selector 104 analyzes the feature information, thereby recommending a hospital and a department of the hospital for treating the patient. The transmitter 105 transmits the recommendation (the hospital and the department of the hospital for treating the patient). e.g., via internet or a wireless communication means, to the terminal of the patient, thereby facilitating the patient to make an appointment with the recommended hospital and department.

The present apparatus for automatically triaging the patient is capable of receiving patient information remotely, accurately analyzing the received patient information, providing a recommendation on the hospital and the department for treating the patient, and transmitting the recommendation remotely to the patient. The present apparatus facilitates the patient to determine the appropriate hospital and department for getting prompt and accurate diagnosis and treatment, avoiding incorrect diagnosis and treatment, wastes of medical resources, and delay of treatment due to inaccurate triage.

In some embodiments, the apparatus further includes a data base storing a plurality of reference feature information. The selector 104 is configured to compare the feature information extracted from the patient information with the plurality of reference feature information, and provide the recommendation based on a result of comparing by the selector 104. Optionally, the plurality of reference feature information includes a plurality of reference feature information of patients treated by a plurality of hospitals and a plurality of departments of the plurality of hospitals. The selector 104 is configured to select one of the plurality of reference feature information of patients treated in one of the plurality of hospitals and one of the plurality of departments as a closest match to the feature information extracted from the patient information, and recommend a selected hospital and a selected department having the closest match for treating the patient.

Optionally, a closer match between the feature information and one of the plurality of reference feature information indicates a higher probability that the one of the plurality of hospitals and the one of the plurality of departments is suitable for diagnosing or treating the patient. Optionally, the selector 104 is configured to select the closest match as the recommendation to the patient. Optionally, the selector 104 is configured to select a plurality of closest matches as the recommendation to the patient.

In some embodiments, the patient information comprises an image of a body part of the patient. The feature extractor 103 is configured to extract a feature partial image information from the image of the body part of the patient as the feature information. Optionally, the step of extracting the feature partial image information is performed by an image recognition technique. Optionally, the plurality of reference feature information includes a plurality of reference feature partial image information. Optionally, the plurality of reference feature partial image information comprise feature partial image information extracted from images of body parts of patients treated in the plurality of hospitals and the plurality of departments. The selector 104 is configured to select one of the plurality of reference feature partial image information as the closest match to the feature partial image information, and recommend the selected hospital and the selected department having the closest match for treating the patient. Optionally, the selector 104 is configured to select the closest match using a classification algorithm. Optionally, the classification algorithm includes one or a combination of a k-means algorithm, and a learning vector quantization-based neural network classification algorithm.

Examples of images of body parts of patients include tongue images, X-ray images, and computed tomography images. In one example, the feature extractor 103 extracts a feature partial image information from the image (tongue images, X-ray images, or computed tomography images) of the body part of the patient as the feature information, the selector 104 compares the feature partial image information extracted by the feature extractor 103 with the plurality of reference feature partial image information (e.g., feature partial image information extracted from images of body parts of patients treated in the plurality of hospitals and the plurality of departments), the selector 104 next selects one of the plurality of reference feature partial image information as the closest match to the feature partial image information using a classification algorithm (such as a k-means algorithm, and a learning vector quantization-based neural network classification algorithm), the selector 104 then recommends the selected hospital and the selected department having the closest match for treating the patient.

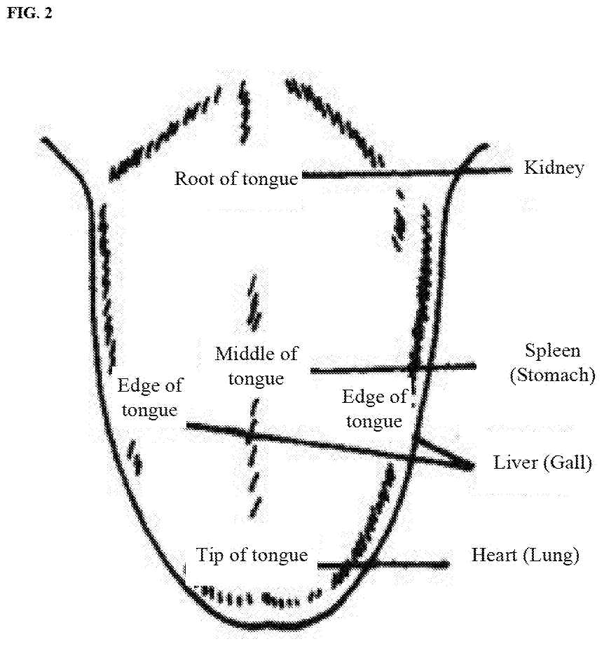

FIG. 2 depicts a tongue image and disease correlation with feature information of different parts of the tongue image. Referring to FIG. 2, the tongue includes at least four parts, including root of tongue, middle of tongue, edge of tongue, and tip of tongue. The abnormalities in the root of tongue typically relates to kidney diseases, the abnormalities in the middle of tongue typically relates to spleen diseases and stomach diseases, the abnormalities in the edge of tongue typically relates to liver diseases and gall diseases, and the abnormalities in the tip of tongue typically relates to heart diseases and lung diseases. Accordingly, the feature extractor 103 is capable of respectively extracting feature partial image information from the root of tongue, the middle of tongue, the edge of tongue, and the tip of tongue. The data base of the apparatus stores a plurality of reference feature partial image information respectively corresponding to the root of tongue, the middle of tongue, the edge of tongue, and the tip of tongue. In one example, the color of the tongue may be used as feature partial image information. In one example, a tongue having a pinky color indicates a healthy state or condition. In another example, a tongue having a color other than a pinky color (e.g., a pale white color, a red color, a purple-red color, a purple color, and a cyan color) indicates an unhealthy or diseased state or condition. Optionally, the data base further stores information regarding suitable hospitals and departments for treating diseases and conditions corresponding to tongues having various colors other than the pinky color.

In some embodiments, the selector 104 selects one of the plurality of reference feature partial image information as the closest match to the feature partial image information using a classification algorithm (such as a k-means algorithm, and a learning vector quantization-based neural network classification algorithm). In one example, a patient's tongue image indicates feature partial image information of the root of tongue has a closest match with a reference partial image information with a pinky color, feature partial image information of the middle of tongue has a closest match with a reference partial image information with a pinky color, and feature partial image information of the tip of tongue has a closest match with a reference partial image information with a purple color. This indicates abnormalities associated with heart diseases. Accordingly, the select 104 recommends hospitals and departments specializing heart diseases and cardiology to the patient.

In some embodiments, the patient information includes an image of a diagnostic textual data. The feature extractor 103 is configured to recognize a textual data from the image of the diagnostic textual data using a textual recognition technique, and extract a semantic feature information from the textual data as the feature information using a semantic analysis technique. Optionally, the plurality of reference feature information include a plurality of reference semantic feature information. Optionally, the plurality of reference semantic feature information include semantic feature information extracted from diagnostic textual data of patients treated in the plurality of hospitals and the plurality of departments. The selector 104 is configured to select one of the plurality of reference semantic feature information as the closest match to the semantic feature information, and recommend the selected hospital and the selected department having the closest match for treating the patient. Optionally, extracting the semantic feature information is performed by a natural language processing technique.

Examples of images of the diagnostic textual data include images of physical examination reports and images of clinical laboratory test reports. In one example, the feature extractor 103 recognizes a textual data from the image of the diagnostic textual data (e.g., images of physical examination reports and images of clinical laboratory test reports) using a textual recognition technique. For example, the feature extractor 103 can first convert the image into a word document, and then extracting the semantic feature information in the word document using a natural language processing technique. Examples of the semantic feature information include an electrocardiogram test indication of a normal condition, an electrocardiogram test indication of an abnormal condition, a Hepatitis B five items test indication of a positive result, and a Hepatitis B five items test indication of a negative result.

The selector 104 compares the semantic feature information extracted by the feature extractor 103 with the plurality of reference semantic feature information (e.g., semantic feature information extracted images of the diagnostic textual data of patients treated in the plurality of hospitals and the plurality of departments), the selector 104 next selects one of the plurality of reference semantic feature information as the closest match to the semantic feature information using a natural language processing technique, the selector 104 then recommends the selected hospital and the selected department having the closest match for treating the patient. In one example, the semantic feature information extracted by the feature extractor 103 includes an electrocardiogram test indication of an atrioventricular block condition, the selector 104 recommends to the patient a hospital and a department that specializes in cardiovascular diseases for further diagnosis and treatment. In another example, the semantic feature information extracted by the feature extractor 103 includes a Hepatitis B five items test indication of a positive result of Hepatitis B surface antigen test, a positive result of Hepatitis B virus e antigen test, a positive result of Hepatitis B virus core antigen test, the selector 104 recommends to the patient a hospital and a department that specializes in liver diseases for further diagnosis and treatment.

In some embodiments, the apparatus further includes a question generator configured to generate a health information query and send the health information query to the terminal 102. The receiver 101 is configured to receive an answer to the health information query from the terminal 102 as the patient information of the patient. In one example, the question generator asks one or more questions such as “Do you have a fever?”, “Do you have diarrhea?”, and “Do you have any body ache?”, and so on. The receiver 101 receives the answers to these questions from the patient, and use the answers as the patient information. The apparatus recommends the hospital and department for further diagnosis and treatment based on these answers. In one example, the answers include “Yes, I have a stomachache.” The apparatus accordingly recommends to the patient a hospital and a department that specializes in treating stomach diseases.

In some embodiments, the selector 104 is configured to provide the recommendation on a plurality of hospitals and a plurality of departments thereof for treating the patient, and rank the plurality of hospitals and the plurality of departments. The transmitter 105 is configured to transmit to the terminal information on one or more highest-ranking hospitals and one or more highest-ranking departments as the recommendation. Optionally, the transmitter 105 is configured to transmit to the terminal information on a single highest-ranking hospital and a single highest-ranking department as the recommendation.

In some embodiments, the selector 104 selects multiple hospitals and departments, all of which are suitable for diagnosing and treating the patient, e.g., multiple highest-ranking hospitals and departments. The selector 104 can recommend all of the multiple hospitals and departments with a ranking order of these multiple hospitals and departments based on the closeness of the match between the feature information and the reference feature information. Other factors may be considered in performing the ranking. Examples of these factors include levels of standard of care of the multiple hospitals and departments, levels of medical expenses of the multiple hospitals and departments, vacancies of the multiple hospitals and departments, and the distances of the multiple hospitals and departments to the patient's location.

Optionally, the transmitter 105 transmits information on only a single highest-ranking hospital and a single highest-ranking department to the patient. Optionally, the transmitter 105 transmits information on multiple highest-ranking hospitals and multiple highest-ranking departments to the patient, and the patient may select one of them based on the provided information.

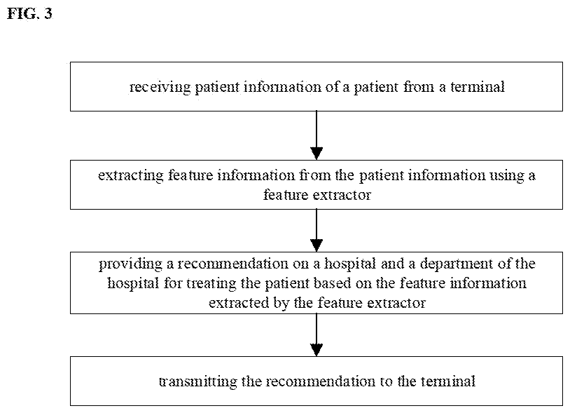

In another aspect, the present disclosure provides an automatic triage method. FIG. 3 is a flow chart illustrating an automatic triage method in some embodiments according to the present disclosure. Referring to FIG. 3, the method in some embodiments includes receiving patient information of a patient from a terminal; extracting feature information from the patient information using a feature extractor; providing a recommendation on a hospital and a department of the hospital for treating the patient based on the feature information extracted by the feature extractor; and transmitting the recommendation to the terminal.

In some embodiments, the patient information of the patient is received by a wire or wirelessly. e.g., via internet or a wireless communication means (e.g., Bluetooth), for example, from a computer or a mobile phone of the patient.

In some embodiments, the automatic triage method further includes storing a plurality of reference feature information; comparing the feature information extracted from the patient information with the plurality of reference feature information; and providing the recommendation based on a result of comparing. A closer match between the feature information and one of the plurality of reference feature information indicates a higher probability that the one of the plurality of hospitals and the one of the plurality of departments is suitable for diagnosing or treating the patient. Optionally, the method includes selecting the closest match as the recommendation to the patient. Optionally, the method includes selecting a plurality of closest matches as the recommendation to the patient.

In some embodiments, the recommendation (the hospital and the department of the hospital for treating the patient) is transmitted via internet or a wireless communication means, to the terminal of the patient, thereby facilitating the patient to make an appointment with the recommended hospital and department.

The present automatic triage method is capable of receiving patient information remotely, accurately analyzing the received patient information, providing a recommendation on the hospital and the department for treating the patient, and transmitting the recommendation remotely to the patient. The present method facilitates the patient to determine the appropriate hospital and department for getting prompt and accurate diagnosis and treatment, avoiding incorrect diagnosis and treatment, wastes of medical resources, and delay of treatment due to inaccurate triage.

In some embodiment, the plurality of reference feature information include a plurality of reference feature information of patients treated by a plurality of hospitals and a plurality of departments of the plurality of hospitals. Optionally, the method further includes selecting one of the plurality of reference feature information of patients treated in one of the plurality of hospitals and one of the plurality of departments as a closest match to the feature information extracted from the patient information; and recommending a selected hospital and a selected department having the closest match for treating the patient.

In some embodiments, the patient information includes an image of a body part of the patient, and the plurality of reference feature information include a plurality of reference feature partial image information. Optionally, the method further includes extracting a feature partial image information from the image of the body part of the patient as the feature information; selecting one of the plurality of reference feature partial image information as the closest match to the feature partial image information; and recommending the selected hospital and the selected department having the closest match for treating the patient. Optionally, the plurality of reference feature partial image information include feature partial image information extracted from images of body parts of patients treated in the plurality of hospitals and the plurality of departments.

In some embodiments, the step of extracting the feature partial image information is performed by an image recognition technique, and the step of selecting the closest match is performed using a classification algorithm. Optionally, the classification algorithm includes one or a combination of a k-means algorithm, and a learning vector quantization-based neural network classification algorithm.

In some embodiments, the patient information includes an image of a diagnostic textual data, and the plurality of reference feature information include a plurality of reference semantic feature information. Optionally, the method further includes recognizing a textual data from the image of the diagnostic textual data using a textual recognition technique; extracting a semantic feature information from the textual data as the feature information using a semantic analysis technique; select one of the plurality of reference semantic feature information as the closest match to the semantic feature information; and recommending the selected hospital and the selected department having the closest match for treating the patient. Optionally, the plurality of reference semantic feature information include semantic feature information extracted from diagnostic textual data of patients treated in the plurality of hospitals and the plurality of departments. Optionally, the step of extracting the semantic feature information is performed by a natural language processing technique.

In some embodiments, the automatic triage method further includes generating a health information query; sending the health information query to the terminal; and receiving an answer to the health information query from the terminal as the patient information of the patient.

In some embodiments, the step of providing the recommendation includes providing the recommendation on a plurality of hospitals and a plurality of departments thereof for treating the patient, and ranking the plurality of hospitals and the plurality of departments. Optionally, the step of transmitting the recommendation to the terminal includes transmitting to the terminal information on one or more highest ranking hospitals and one or more highest ranking departments as the recommendation.

The foregoing description of the embodiments of the invention has been presented for purposes of illustration and description. It is not intended to be exhaustive or to limit the invention to the precise form or to exemplary embodiments disclosed. Accordingly, the foregoing description should be regarded as illustrative rather than restrictive. Obviously, many modifications and variations will be apparent to practitioners skilled in this art. The embodiments are chosen and described in order to explain the principles of the invention and its best mode practical application, thereby to enable persons skilled in the art to understand the invention for various embodiments and with various modifications as are suited to the particular use or implementation contemplated. It is intended that the scope of the invention be defined by the claims appended hereto and their equivalents in which all terms are meant in their broadest reasonable sense unless otherwise indicated. Therefore, the term “the invention”, “the present invention” or the like does not necessarily limit the claim scope to a specific embodiment, and the reference to exemplary embodiments of the invention does not imply a limitation on the invention, and no such limitation is to be inferred. The invention is limited only by the spirit and scope of the appended claims. Moreover, these claims may refer to use “first”, “second”, etc. following with noun or element. Such terms should be understood as a nomenclature and should not be construed as giving the limitation on the number of the elements modified by such nomenclature unless specific number has been given. Any advantages and benefits described may not apply to all embodiments of the invention. It should be appreciated that variations may be made in the embodiments described by persons skilled in the art without departing from the scope of the present invention as defined by the following claims. Moreover, no element and component in the present disclosure is intended to be dedicated to the public regardless of whether the element or component is explicitly recited in the following claims.

1. An apparatus for automatically triaging a patient, comprising:
a receiver configured to receive patient information of the patient from a terminal;
a feature extractor configured to extract feature information from the patient information;
a selector configured to provide a recommendation on a hospital and a department of the hospital for treating the patient based on the feature information extracted by the feature extractor; and
a transmitter configured to transmit the recommendation to the terminal.

2. The apparatus of claim 1, further comprising a data base storing a plurality of reference feature information;
wherein the selector is configured to compare the feature information extracted from the patient information with the plurality of reference feature information, and provide the recommendation based on a result of comparing by the selector.

3. The apparatus of claim 2, wherein the plurality of reference feature information comprises a plurality of reference feature information of patients treated by a plurality of hospitals and a plurality of departments of the plurality of hospitals;
the selector is configured to select one of the plurality of reference feature information of patients treated in one of the plurality of hospitals and one of the plurality of departments as a closest match to the feature information extracted from the patient information, and recommend a selected hospital and a selected department having the closest match for treating the patient.

4. The apparatus of claim 3, wherein the patient information comprises an image of a body part of the patient;
the feature extractor is configured to extract a feature partial image information from the image of the body part of the patient as the feature information;
the plurality of reference feature information comprise a plurality of reference feature partial image information; and
the selector is configured to select one of the plurality of reference feature partial image information as the closest match to the feature partial image information, and recommend the selected hospital and the selected department having the closest match for treating the patient.

5. (canceled)

6. The apparatus of claim 4, wherein extracting the feature partial image information is performed by an image recognition technique; and
the selector is configured to select the closest match using a classification algorithm.

7. The apparatus of claim 6, wherein the classification algorithm comprises one or a combination of a k-means algorithm, and a learning vector quantization-based neural network classification algorithm.

8. The apparatus of claim 3, wherein the patient information comprises an image of a diagnostic textual data;
the feature extractor is configured to recognize a textual data from the image of the diagnostic textual data using a textual recognition technique, and extract a semantic feature information from the textual data as the feature information using a semantic analysis technique;
the plurality of reference feature information comprise a plurality of reference semantic feature information; and
the selector is configured to select one of the plurality of reference semantic feature information as the closest match to the semantic feature information, and recommend the selected hospital and the selected department having the closest match for treating the patient.

9. (canceled)

10. The apparatus of claim 8, wherein extracting the semantic feature information is performed by a natural language processing technique.

11. The apparatus of claim 1, further comprising a question generator configured to generate a health information query and send the health information query to the terminal;
wherein the receiver is configured to receive an answer to the health information query from the terminal as the patient information of the patient.

12. The apparatus of claim 1, wherein the selector is configured to provide the recommendation on a plurality of hospitals and a plurality of departments thereof for treating the patient, and rank the plurality of hospitals and the plurality of departments; and
the transmitter is configured to transmit to the terminal information on one or more highest-ranking hospitals and one or more highest-ranking departments as the recommendation.

13. An automatic triage method, comprising:
receiving patient information of a patient from a terminal;
extracting feature information from the patient information using a feature extractor;
providing a recommendation on a hospital and a department of the hospital for treating the patient based on the feature information extracted by the feature extractor; and
transmitting the recommendation to the terminal.

14. The automatic triage method of claim 13, further comprising:
storing a plurality of reference feature information;
comparing the feature information extracted from the patient information with the plurality of reference feature information; and
providing the recommendation based on a result of comparing.

15. The automatic triage method of claim 14, wherein the plurality of reference feature information comprises a plurality of reference feature information of patients treated by a plurality of hospitals and a plurality of departments of the plurality of hospitals;
the method further comprises selecting one of the plurality of reference feature information of patients treated in one of the plurality of hospitals and one of the plurality of departments as a closest match to the feature information extracted from the patient information; and
recommending a selected hospital and a selected department having the closest match for treating the patient.

16. The automatic triage method of claim 15, wherein the patient information comprises an image of a body part of the patient; and
the plurality of reference feature information comprise a plurality of reference feature partial image information;
the method further comprises extracting a feature partial image information from the image of the body part of the patient as the feature information;
selecting one of the plurality of reference feature partial image information as the closest match to the feature partial image information; and
recommending the selected hospital and the selected department having the closest match for treating the patient.

17. (canceled)

18. The automatic triage method of claim 16, wherein extracting the feature partial image information is performed by an image recognition technique; and
selecting the closest match is performed using a classification algorithm.

19. The automatic triage method of claim 18, wherein the classification algorithm comprises one or a combination of a k-means algorithm, and a learning vector quantization-based neural network classification algorithm.

20. The automatic triage method of claim 15, wherein the patient information comprises an image of a diagnostic textual data; and
the plurality of reference feature information comprise a plurality of reference semantic feature information;
the method further comprises recognizing a textual data from the image of the diagnostic textual data using a textual recognition technique;
extracting a semantic feature information from the textual data as the feature information using a semantic analysis technique;
selecting one of the plurality of reference semantic feature information as the closest match to the semantic feature information; and
recommending the selected hospital and the selected department having the closest match for treating the patient.

21. (canceled)

22. The automatic triage method of claim 20, wherein extracting the semantic feature information is performed by a natural language processing technique.

23. The automatic triage method of claim 13, further comprising generating a health information query;
sending the health information query to the terminal; and
receiving an answer to the health information query from the terminal as the patient information of the patient.

24. The automatic triage method of claim 13, wherein
providing the recommendation comprises providing the recommendation on a plurality of hospitals and a plurality of departments thereof for treating the patient, and ranking the plurality of hospitals and the plurality of departments; and
transmitting the recommendation to the terminal comprises transmitting to the terminal information on one or more highest-ranking hospitals and one or more highest-ranking departments as the recommendation.
